(19) United States
(12) Patent Application Publication
(10) Pub. No.: US 2010/0286775 A1
(43) Pub. Date: Nov. 11, 2010

(54) Ligament and Tendon Prosthesis

(75) Inventor: Idan Tobis, Beit Hashmonai (IL)

Correspondence Address: GREENBERG TRAURIG, LLP, MET LIFE BUILDING, 200 PARK AVENUE, NEW YORK, NY, 10166, US

(73) Assignee: TAVOR [I.T.N] LTD.,, Ashqelon (IL)

(21) Appl. No.: 12/682,725

(22) PCT Filed: Oct. 12, 2008
(86) PCT No.: PCT/IL08/01346

§ 371 Date: Jul. 9, 2010

Related U.S. Application Data
(60) Provisional application No. 60/960,732, filed on Oct. 11, 2007.

(51) Int. Cl. A61F 2/08; B23P 11/00

(52) U.S. Cl. 623/13.12; 623/13.15; 623/13.11; 29/428

(57) ABSTRACT

The invention provides a ligament or tendon prosthesis. The prosthesis has at least two load bearing elements (6a, 6b) arranged in series and having different mechanical properties. The invention also provides a method for producing the prosthesis of the invention, as well as a method for replacing a torn or damaged ligament or tendon.

### FIELD OF THE INVENTION

This invention relates to medical devices, and more specifically to such devices for treating a torn or otherwise damaged ligament or tendon.

### BACKGROUND OF THE INVENTION

The human and animal body contains numerous tendons and ligaments. Although tendons and ligaments have similar anatomical structures, they serve different biological function. Both serve as load-bearing structures, with tendons attaching muscle to bone, while ligaments attach bone to bone.

Ligament and tendon injuries are very common in sports that require stopping 10 and starting or quickly changing directions. Under such conditions, the extreme forces on the knee, for example, can result in torn ligaments. The anterior cruciate ligament (ACL) and the medial collateral ligament (MCL) are the most often injured, but the posterior cruciate ligament (PCL) and the lateral collateral ligament (LCL) can also be injured.

One method for repairing a torn tendon or ligament is to fasten the tendon or ligament ends together by means of a suture. In the case of more extensive injuries where loss of substance must be bridged, a known method of treatment involves transplanting a graft consisting of a ligament or tendon harvested either from another location in the patient's body (an autograft) or from a donor (allograft). Although this process is often successful, it results in loss of some degree of mobility in the donor location, as well as various other complications such as pain and local morbidity at the donor site in the case of an autograft, and a risk of infection in the case of an allograft.

Another method for treating a torn ligament or tendon is to introduce a prosthesis to replace the torn ligament or tendon. Such prostheses are typically formed as a bundle of loosely bundled fibers or a coreless tubular structure.

The material of the prosthesis must be compatible with other body tissues and at the same time resist abrasion in response to movement of bone against the surface of the prosthesis. In recent years, prostheses of materials without adverse tissue reaction, such as Dacron, Teflon and polypropene, have been used to a certain extent for replacing tendons and ligaments and (amongst them, the ACL). While it has been possible to reduce the time that the patient must keep the body part in question immobilized, and to thereby to avoid some of the above mentioned disadvantages, these prostheses have sometimes resulted in the formation of granuloma and incomplete function. In particular, for cruciate ligament prostheses it has not been possible to obtain either a satisfactory stability of the knee joint or the necessary strength of the tissue formed upon healing, and after prolonged use the prostheses have been found to break due to fatigue of the material.

Ligament and tendon prostheses are disclosed, for example, in U.S. Pat. Nos. 4,642,119; 6,599,319; 4,792,336; 6,287,340; 5,595,621; 5,575,819; 7,101,398; 4,755,183; 4,932,972; 3,953,896, 3,545,008, 4,187,558, 3,797,047, 3,805,300, 5,004,474, and 5,197,983.

The following publications are considered as being of significance for an understanding of the invention:

Brown, T D, et al., Acta orthop. Scand 51, 429-437, 1980.

Noyes F R, et al, Journal of Bone and Joint Surgery, 66A (3), 344-352, 1984.

Shabolovskaya, S. Biomedical Materials and Engineering, 6 (A), 267-289, 1996.

### SUMMARY OF THE INVENTION

The present invention provides a ligament or tendon prosthesis for use in replacing a torn or otherwise damaged, ligament or tendon, such as an anterior cruciate Ligament (ACL). The prosthesis of the invention may be used to replace a ligament or tendon in a human or any animal such as a horse, dog, cat, pig, cattle.

The prosthesis of the invention is generally elongated in shape. Each end of the prosthesis is adapted for attachment to a musco-skeletal tissue such as bone. The prosthesis comprises at least a first load bearing element and a second load bearing element, where the first and second load bearing elements are arranged in series. The first and second load bearing elements have different mechanical properties. For example, the two load bearing elements may be made from different materials having different elastic coefficients (the ratio of the tension to strain). As another example, the two load bearing elements may be made from the same material, but differently shaped so as to have different effective elastic coefficients, resulting from their different bending moments.

The shape of the load bearing elements are preferably selected in order to impart to the prosthesis the general shape of the tendon or ligament the prosthesis is designed to replace. According to the application, the load bearing elements may be, for example, flat, circular, elliptical, double-circular, or triple-circular. The mechanical properties of the load bearing elements are preferably selected to impart to the prosthesis a yield force greater than or equal to that of the ligament or tendon the prosthesis is to replace.

In a presently preferred embodiment, the prosthesis comprises three load bearing elements arranged in series. The central load bearing element preferably has mechanical properties approximating those of the ligament or tendon that the prosthesis is to replace. The two terminal load bearing elements preferably have mechanical properties approximating those of the musco-skeletal tissue to which they are attached. Thus, in a preferred embodiment, the terminal load bearing elements are stiffer or less compliant than the central load bearing element. For example, the central load bearing element may have a coefficient of elasticity that is smaller than coefficients of elasticity of the terminal load bearing elements. The mechanical properties of various connective tissues, such as bone, ligaments and tendons, can be found, for example, in the publications of Brown, Noyes, and Shabolovskaya cited above.

In one embodiment, the prosthesis comprises a plate or cylindrical rod, in which one or more holes, grooves or slots have been formed in a first region of the plate or rod surface. The size and shape of the holes, grooves or slots in the first region are selected to impart to that region of the plate mechanical properties approximating those of the tendon or ligament the prosthesis is to replace. A second region of the plate is either formed with holes, grooves, or slots of a different shape or size that those in the first region, or is devoid of such holes, grooves or slots, so as to have mechanical properties approximating those of the musco-skeletal tissue to which the prosthesis is attached.

In another embodiment of the invention, the prosthesis comprises two rods arranged longitudinally in series. The two rods may be made from different materials, and may have different dimensions so that the two rods have different mechanical properties.

The load bearing elements may be made form an alloy. The alloy may be, for example, stainless steel, such as stainless steel 316L or 316 LVM. The alloy may also be a cobalt based alloy, such as ASTM F75, ASTM F799, ASTM F790 or ASTM F562. The alloy may also be a titanium based alloy, such as ASTM F136. The alloy may also exhibit super elastic properties at body temperature, or under stress, such as exhibited by Nitinol™.

The prosthesis of the invention may comprise a sleeve containing the load bearing elements in order to protect nearby organs and tissues from abrasion from the prosthesis and vice versa, as the prosthesis moves in response to movement of the musco-skeletal tissues to which the prosthesis is connected. The sleeve may be made, for example, from biodegradeable polymers such as polyglecaprone, polyglycolic acid, polylactyc acid, polydioxanone, or co-polymers of the aforementioned polymers. According to still further features in the described preferred embodiments the device further comprises a sleeve made of a polysaccharide such as Chondroitin sulphate or hyaluronic acid. The sleeve may be made of a synthetic polymer based on hyaluronic acid such as HYAFF™.

Thus, in its first aspect, the present invention provides a ligament or tendon prosthesis having a first end and a second end, comprising:
(a) a first load bearing element; and
(b) a second load bearing element, the first and second load bearing elements differing in one or more mechanical properties;
wherein the first and second load bearing elements are arranged in the prosthesis in series.

In its second aspect, the invention provides a method for producing prosthesis for replacing a ligament or tendon, comprising:
(a) providing a first load bearing element and a second load bearing element, the first and second load bearing elements differing in one or more mechanical properties; and
(b) joining the first and second Toad bearing elements together in series.

In another of its aspects, the invention provides a method for replacing a torn or damaged tendon or ligament, comprising:
(a) providing a ligament or tendon prosthesis according to any one of claims 1 to 11;
(b) attaching the first end of the prosthesis to a musco-skeletal tissue to which the tendon or ligament is attached; and
(c) attaching the second end of the prosthesis to a musco-skeletal tissue to which the tendon or ligament is attached.

In yet another of its aspects, the invention provides a method for replacing a torn or damaged tendon or ligament in a non-human animal, comprising:
(a) providing a ligament or tendon prosthesis according to any one of claims 1 to 11;
(b) attaching the first end of the prosthesis to a musco-skeletal tissue of the animal to which the tendon or ligament is attached; and
(c) attaching the second end of the prosthesis to a musco-skeletal tissue to which the tendon or ligament is attached.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 shows the general conceptual composition of a tendon or ligament prosthesis in accordance with the invention;

FIG. 2 shows a tendon or ligament prosthesis formed from a plate, in accordance with one embodiment of the invention;

FIG. 3 shows a tendon or ligament prosthesis formed from a cylinder or rod, in accordance with a second embodiment of the invention;

FIG. 4 shows a tendon or ligament prosthesis formed from three cylinders or rods, in accordance with a third embodiment of the invention;

FIG. 5 shows a tendon or ligament prosthesis comprising wires, in accordance with a fourth embodiment of the invention in a side view (5a) and in a top view (5b); and

FIG. 6 shows the prosthesis of FIG. 5 after implantation in a knee joint.

### DETAILED DESCRIPTION OF EMBODIMENTS

It is to be understood that the foregoing drawings, and the description below, are provided primarily for purposes of facilitating understanding the conceptual aspects of the invention and possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein

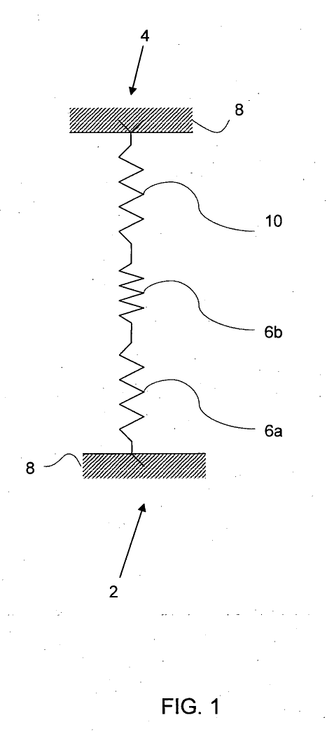

FIG. 1 illustrates schematically a ligament or tendon prosthesis 1 in accordance with one embodiment of the invention. The prosthesis 1 is generally elongated in shape and has a first end 2 and a second end 4. The first and second ends are adapted for attachment to musco-skeletal tissue 8 in the body, such as bone, as described below. The prosthesis 1 comprises two load bearing elements 6a and 6b. The load bearing elements 6a and 6b are arranged in series. The first and second load bearing elements have different mechanical properties. For example, the load bearing elements 6a and 6b may be made from different materials having different elastic coefficients (the ratio of the tension to strain). As another example, the first and second load bearing elements may be made from the same material, but differently shaped so as to have different bending moments. The prosthesis 1 may include additional load bearing elements, such as the load bearing element 10. One additional load bearing element 10 is shown in FIG. 1. This is by way of example only, and the prosthesis 1 can comprise any number of additional load bearing elements as required in any application. When present, the additional load bearing elements may be positioned in series or in parallel with the first and second load bearing elements 6a and 6b. The first and second load bearing elements may be adjacent to each other in the prosthesis, as shown in FIG. 1, or they may be separated by an additional intervening load bearing element.

Preferably, the mechanical properties of the load bearing elements are selected in order to impart to the prosthesis 1 mechanical properties similar to those of the tendon or ligament that prosthesis 1 is to replace. In particular, the mechanical properties of the load bearing element are selected to impart to the prosthesis a stress (tension)-strain curve resembling that of a natural ligament, with a yield force greater than or equal to that of the ligament or tendon the prosthesis is to replace.

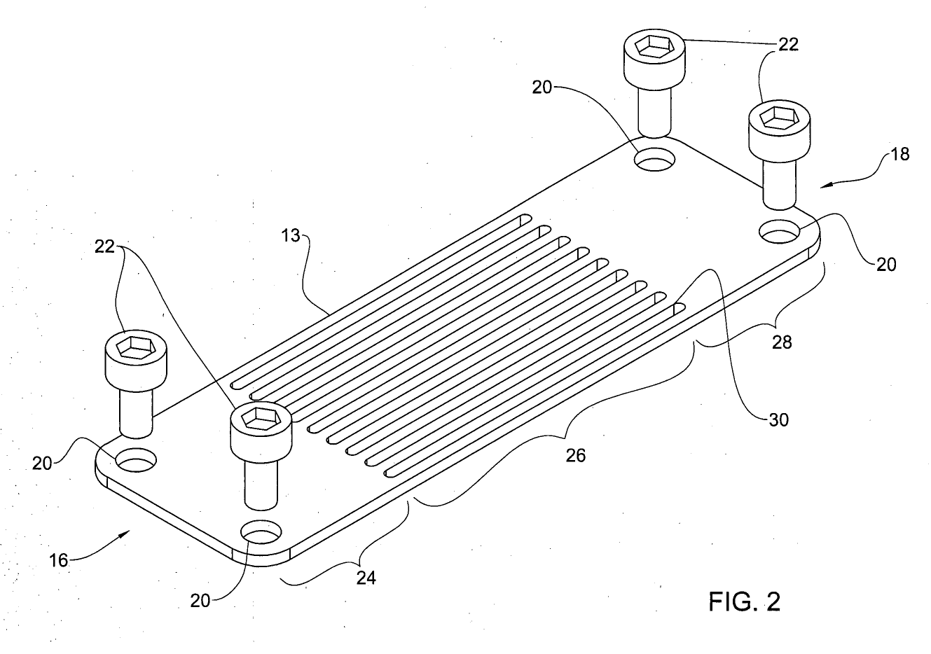

FIG. 2 shows a tendon or ligament prosthesis 13 in accordance with one embodiment of the invention. The prosthesis 13 comprises an elongated plate 11 having a first end 16 and a second end 18. The first and second ends 16 and 18 are provided with holes 20 for receiving a screw 22 adapted for affixing the first and second ends to musco-skeletal tissue in the body, such as bone. The plate 11 may have a rectangular cross section and may be made, for example, from a metallic material. A portion of the plate 11 adjacent to the first end 16 functions as a first load bearing element 24. A portion of the plate 11 adjacent to the first load bearing element 24 functions as a second load bearing element 26. A third portion of the plate 11 intervening between the second load bearing element 26 and the second end 18 functions as an additional load bearing element 28. The second load bearing element 26 thus intervenes between the first load bearing 24 element and the additional load bearing element 28. The second load bearing element 26 is provided with one or more holes, grooves or slots 30 formed in the plate 11. The holes, grooves, or slots 30 may be straight as shown in FIG. 2, or may have any desired form. The shape and dimensions of the holes, grooves, or slots 30 are selected to impart to the second load bearing element 26 desired mechanical properties, such as a desired bending moment or tension to strain coefficient. The first load bearing element 24 may also be provided with holes, grooves or slots (not shown) that are different in one or more properties from the holes grooves or slots 30 of the second load bearing element 26, for example, in the shape or dimensions of the holes, grooves or slots or in the spacing between the grooves or slots. The additional load bearing element 28 may also be provided with grooves or slots (not shown).

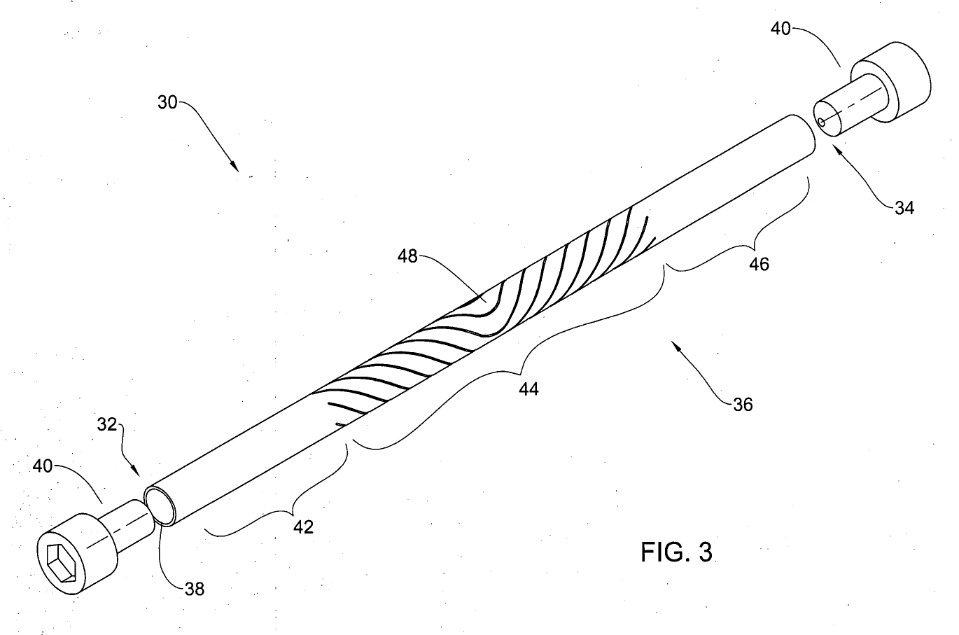

FIG. 3 shows a tendon or ligament prosthesis 30 in accordance with another embodiment of the invention. The prosthesis 30 comprises an elongated cylindrical rod 36 having a first end 32 and a second end 34. The first and second ends 32 and 34 are provided with a hole 38 adapted for receiving a screw 40 for affixing the first and second ends to musco-skeletal tissue in the body, such as bone. The rod 36 may have a circular cross section, as shown in FIG. 3, or any other cross section, as required in any application. The rod 36 may be made, for example, from a metallic material. A portion of the rod 36 adjacent to the first end 32 functions as a first load bearing element 42. A portion of the rod 36 adjacent to the first load bearing element 42 functions as a second load bearing element 44. A third portion of the rod 36 intervening between the second load bearing element 44 and the second end 34 functions as an additional load bearing element 28. The second load bearing element 44 thus intervenes between the first load bearing 42 element and the additional load bearing element 46. The second load bearing element 46 is provided with one or more holes, grooves or slots 48 formed in the rod 36. The holes, grooves, or slots 30 may be curved as shown in FIG. 3, or straight, and may have any desired form. The shape and dimensions of the holes, grooves, or slots 48 are selected to impart to the second load bearing element 44 desired mechanical properties, such as a desired bending moment or tension to strain ratio. Due to the presence of the holes, grooves or slots 48, the bending moment of the second load bearing element 44 is smaller than the bending moment of the first load bearing element 42. The first load bearing element 24 may also be provided with holes, grooves or slots (not shown) that are different in one or more properties from the holes, grooves or slots 48 of the second load bearing element 44, for example, in the shape or dimensions of the holes, grooves or slots or in the spacing between the holes, grooves or slots. The additional load bearing element 46 may also be provided with grooves or slots (not shown).

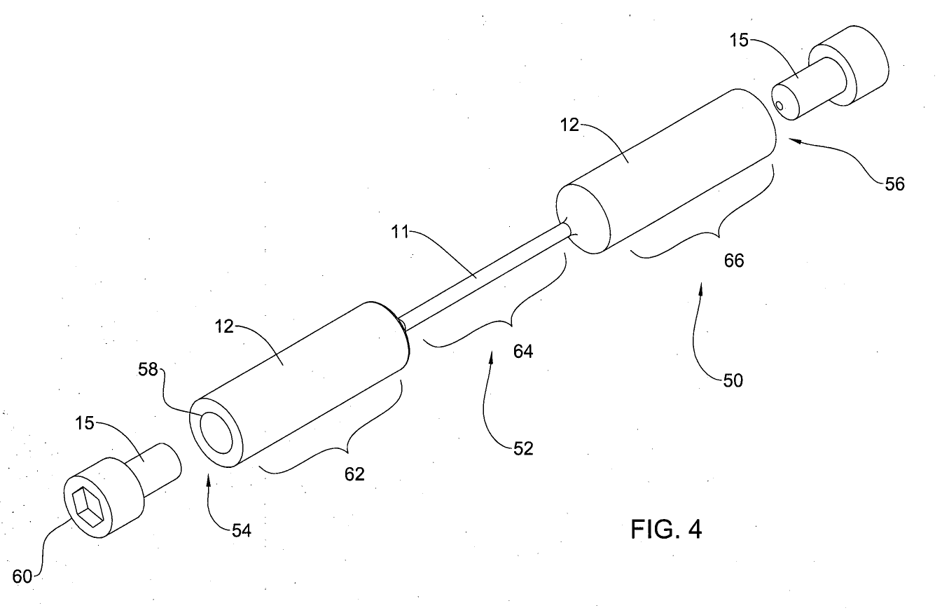

FIG. 4 shows a tendon or ligament prosthesis 50 in accordance with another embodiment of the invention. The prosthesis 50 comprises an elongated portion 52 having a first end 54 and a second end 56. The first and second ends 54 and 56 are provided with a hole 58 adapted for receiving a screw 60 for affixing the first and second ends to musco-skeletal tissue in the body, such as bone. A portion of the elongated portion 52 adjacent to the first end 54 functions as a first load bearing element 62. A portion of the elongated portion 52 adjacent to the first load bearing element 62 functions as a second load bearing element 64. A third portion of the elongated portion 52 intervening between the second load bearing element 64 and the second end 56 functions as an additional load bearing element 66. The second load bearing element 64 thus intervenes between the first load bearing 62 element and the additional load bearing element 66.

A difference in mechanical properties between the first and second load bearing elements 62 and 64 may be achieved by forming the first load bearing element 62 from a material having different mechanical properties from the material used to form the second load bearing element 64. Alternatively, the first and second load bearing elements may be formed from the same material, but having different dimensions (e.g. diameter or thickness). Thus, for example, the first load bearing element 62 may be formed from a cylindrical rod, while the second load bearing element 64 is formed from a thinner rod or wire having a lower modulus of elasticity, than that of the first load bearing element. Alternatively, the first load bearing element 62 may be formed from a n alloy having a relatively high modulus of elasticity such as stainless steel or cobalt based alloys, while the second load bearing element 64 is formed from a relatively thin wire made of a material having a relatively low modulus of elasticity such as titanium or super elastic NiTinol. The first and second load bearing elements preferably have similar galvanic properties. Similarly, the material and dimensions of the additional load bearing element 66 are selected to impart to the additional load bearing element 66 desired mechanical properties.

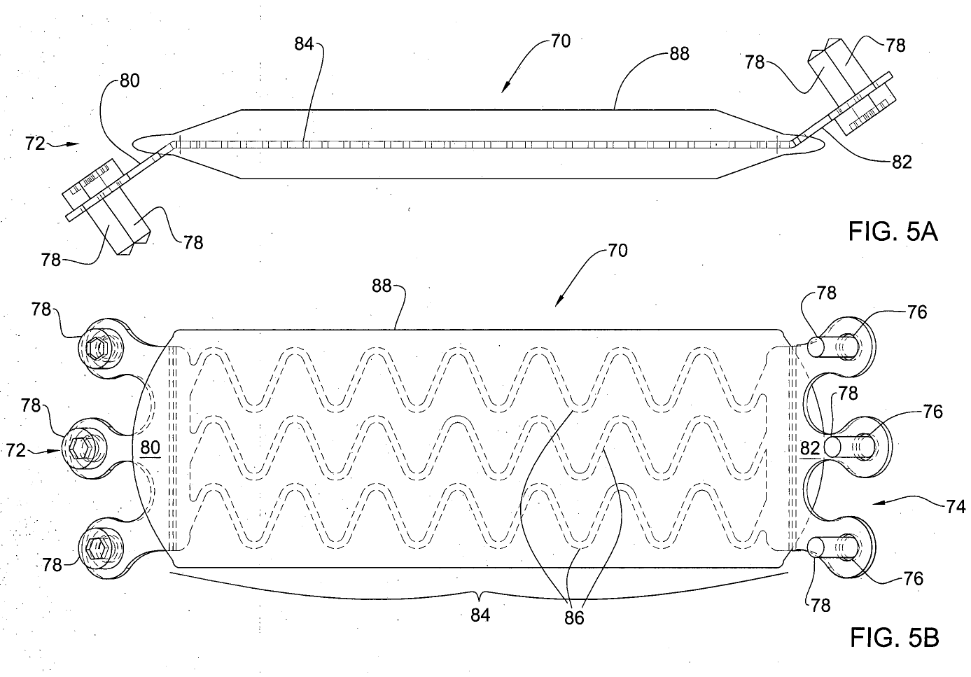

FIG. 5 shows a tendon or ligament prosthesis 70 in accordance with still another embodiment of the invention. The prosthesis 70 is seen in a side view in FIG. 7a, and in a top view in FIG. 7b. The prosthesis 70 has a first end 72 and a second end 74. The first and second ends 72 and 74 are provided with holes 76 for receiving a screw 78 adapted for affixing the first and second ends to musco-skeletal tissue in the body, such as bone. Adjacent to the holes 76 of the first end 72 is a first load bearing element 80. There is a similar load bearing element 82 adjacent to the holes 76 of the second end 74. Intervening between the load bearing elements 80 and 82 is a second load bearing element 84. The second load bearing element 84 is formed by one or more wires 86 connecting the first load bearing element 80 with the load bearing element 82. The second load bearing element 84 has a lower bending moment than that of the first load bearing element 80. The wires 86 may be straight or may have any other shape. In a preferred embodiment, the wires have the shape of a meander, as shown in FIG. 5b. A meander enhances the overall flexibility of the second load bearing element 84, so that the second load bearing element 84 is deformed more easily than the first load bearing element 80. The load bearing elements 80, 82, and 84, are enclosed in a sheath 88, to prevent abrasion between the load bearing elements and surrounding body tissue. The sheath 88 may be made, for example, from a soft polymeric material. The polymer may be bioabsorbable or biodurable.

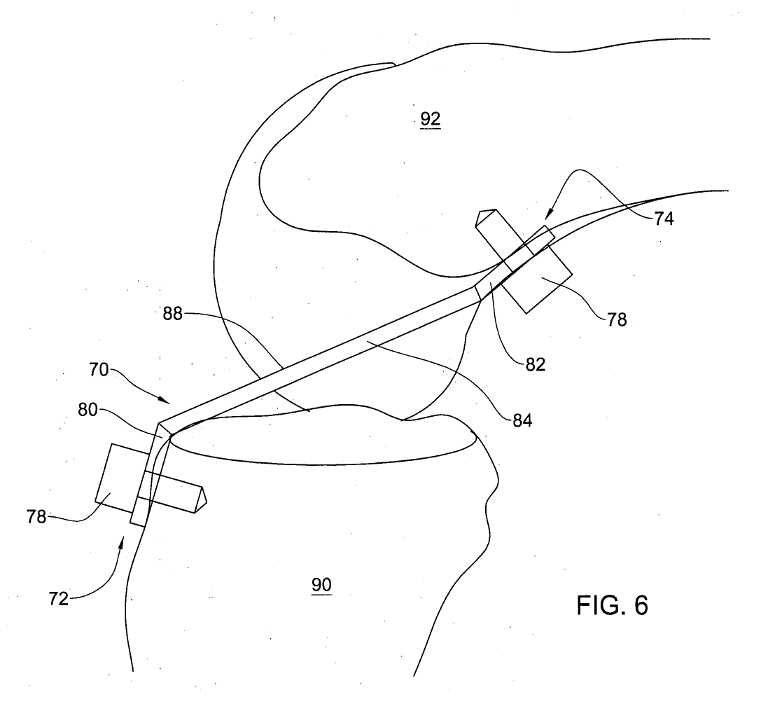

FIG. 6 shows the prosthesis 70 of FIG. 5 after implantation in a knee joint to replace a damaged or missing anterior cruciate ligament (ACL). The first end 72 of the prosthesis has been affixed to a tibia 90 via screws 78 and the second end has been affixed to a femur 92. The load bearing elements 80 and 82, having mechanical properties approximating those of bone, are applied to the tibia 90 and the femur 92, respectively. The second load bearing element 84, having mechanical properties approximating those of the ACL is positioned where the ACL was originally positioned before being damaged. In this configuration, the prosthesis 70 restricts the movement of the bones 90 and 92 in a similar fashion to the functioning of the undamaged ACL.

1. A ligament or tendon prosthesis having a first end and a second end, comprising:
(a) a first load bearing element; and
(b) a second load bearing element, the first and second load bearing elements differing in one or more mechanical properties;
wherein the first and second load bearing elements are arranged in the prosthesis in series.

2. The prosthesis according to claim 1 wherein the first load bearing element is made from a first material having a first elastic coefficient, and the second load bearing elements is made from a second material having a second elastic coefficient, the second elastic coefficient being different from the first elastic coefficient.

3. The prosthesis according to claim 1 wherein the first load bearing element has a first shape and a first set of dimensions, and the second load bearing element has a second shape and a second set of dimensions, at least one of the second shape and second set of dimensions being different from the first shape or the first set of dimensions.

4. The prosthesis according to claim 1 or 3, wherein the first and second load bearing elements are made from the same material.

5. The prosthesis according to any one of the previous claims further comprising a third load bearing element, the third load bearing element being arranged in series with the first load bearing element and the second load bearing element.

6. The prosthesis according to claim 5 wherein the second load bearing element is located in the prosthesis between the first and third load bearing elements, and wherein the second load bearing element has a coefficient of elasticity that is smaller than coefficients of elasticity of the first and third load bearing elements.

7. The prosthesis according to any one of the previous claims configured to replace a torn cruciate ligament.

8. The prosthesis according to any one of the previous claims comprising a plate or cylindrical rod, wherein a first region of the plate or rod is provided with one or more holes, grooves or slots, the first region serving as the first load bearing element.

9. The prosthesis according to claim 8 wherein a second region of the plate or rod is either devoid of holes grooves or slots, or is provided with holes grooves or, slots having at least one or both of a size and shape different from a size and shape of the holes, grooves or slots of the first region, the second region serving as the second load bearing element.

10. The prosthesis according to any one of claims 1 to 8 wherein one or more of the load bearing elements are cylindrical rods.

11. The prosthesis according to any one of the previous claims further comprising a sheath enclosing one or more of the load bearing elements.

12. A method for producing prosthesis for replacing a ligament or tendon, comprising:
(a) providing a first load bearing element and a second load bearing element, the first and second load bearing elements differing in one or more mechanical properties; and
(b) joining the first and second load bearing elements together in series.

13. The method according to claim 12 further comprising adapting the prosthesis for attachment to musco-skeletal tissue at two locations of the prosthesis.

14. The method according to 13 wherein the second load bearing element has one or more mechanical properties approximating mechanical properties of the ligament or tendon to be replaced.

15. The method according to any one of claims 12 to 14 wherein the second load bearing element has a coefficient of elasticity that is smaller than coefficients of elasticity of the first load bearing element.

16. The method according to claim 14 or 15 wherein the first load bearing element has one or more mechanical properties approximating mechanical properties of a musco-skeletal tissue to which the first load bearing element is to be attached.

17. The method according to any one of claims 12 to 16 further comprising providing a third load bearing element and joining the third load bearing element in series to the first and second load bearing elements.

18. The method according to claim 16 or 17 wherein the third load bearing element has one or more mechanical properties approximating mechanical properties of a musco-skeletal tissue to which the first load bearing element is to be attached.

19. The method according to any one of claims 12 to 18 further comprising providing a sheath and introducing one or more of the load bearing elements into the sheath.

20. A method for replacing a torn or damaged tendon or ligament, comprising:
(a) providing a ligament or tendon prosthesis according to any one of claims 1 to 11;
(b) attaching the first end of the prosthesis to a musco-skeletal tissue to which the tendon or ligament is attached; and
(c) attaching the second end of the prosthesis to a musco-skeletal tissue to which the tendon or ligament is attached.

21. A method for replacing a torn or damaged tendon or ligament in a non-human animal, comprising:
(a) providing a ligament or tendon prosthesis according to any one of claims 1 to 11;
(b) attaching the first end of the prosthesis to a musco-skeletal tissue of the animal to which the tendon or ligament is attached; and
(c) attaching the second end of the prosthesis to a musco-skeletal tissue to which the tendon or ligament is attached.
